(19) United States
(12) Patent Application Publication
(10) Pub. No.: US 2026/0013815 A1
(43) Pub. Date: Jan. 15, 2026

(54) METHOD FOR VISUALIZING AND NAVIGATING COMPLEX GEOMETRY FOR A MEDICAL PROCEDURE

(71) Applicant: Thalasso Technologies, L.L.C., Carmel, IN (US)

(72) Inventor: Peter T. Zavislak, Carmel, IN (US)

(73) Assignee: Thalasso Technologies, L.L.C., Carmel, IN (US)

(21) Appl. No.: 19/268,646

(22) Filed: Jul. 14, 2025

Related U.S. Application Data
(60) Provisional application No. 63/671,728, filed on Jul. 15, 2024.

(51) Int. Cl. A61B 6/00; A61B 6/46

(52) U.S. Cl. CPC A61B 6/487; A61B 6/461

(57) ABSTRACT

In one embodiment, an imaging system is provided including a reference marker, a medical device, an imaging array, a computer system, and a display system. The reference marker is fixed in a position within the operational area. The medical device is moveable within the operational area and includes a comparison marker. The imaging array observes the position of the reference marker and a position of the comparison marker. The imaging array comprises at least two cameras. The computer system constructs a three-dimensional model of the operational area and defines the position of the reference marker within the operational area using at least two two-dimensional images of the operational area. The display system is in communication with the imaging array and the computer system. The display system creates a display of the three-dimensional model of the operational area and a position of the medical device within the operational area.

### TECHNICAL FIELD

This disclosure relates to medical procedures and medical devices and, in particular, to methods and systems to image and treat portions of a body having complex geometry.

### BACKGROUND

The statements in this section merely provide background information related to the present disclosure and may not constitute prior art.

Medical procedures sometimes require that a medical device, such as a needle, navigate tortuous geometry to reach a treatment area. In such procedures, it may be desirable to get a complete image of the operational area using computed tomography (CT). Furthermore, a three-dimensional (3D) model of the operational area may be created using CT scans to fully understand the geometry of the operational area. However remote hospitals, surgery centers, and physician offices often do not have access to pre-operative or intra-operative CT scanning equipment and must rely on two-dimensional (2D) images taken by a fluoroscope. Constructing a 3D model of the operational area may require CT scanning equipment and extensive computing equipment, which may be costly and resource intensive, limiting their use to larger metropolitan hospitals. Additionally, excessive radiation exposure can be an issue in many procedures. CT scans typically generate hundreds or thousands of X-ray images, utilizing a substantial amount of X-ray radiation. Furthermore, continuous use of fluoroscopy during the procedures exposes patients to excessive X-ray radiation. Therefore, it is desirable that the method of quickly creating a 3D model of the operational area utilizing only equipment that would be available even in remote locations. Furthermore, it is desirable to have an imaging system which can minimize radiation exposure to a patient before and during a procedure.

Additionally, medical procedures involving percutaneous diagnostics and treatments are often performed in areas of a body having complex geometry, such as the spine. In such procedures, small-caliber metal cannulas may be used to teach the treatment area. However, flexibility of the cannula and tortuous geometry of the path to the treatment area may cause the cannula to be misdirected while advancing to the treatment area. Misdirection of the cannula may cause damage to the tissue around the treatment area. Furthermore, even when a 3D model of the operational area is available, the position of the cannula within the tissue may be difficult to determine, especially in portions of the body having a tortuous geometry. Therefore, it is desirable to have a medical device for such treatments which is small-caliber but reinforced to navigate complex geometry without misdirection. Further, it would be desirable to integrate the position of the medical device into the 3D model of the operational area to determine the position of the device as it is being advanced to the treatment area.

### SUMMARY

Further areas of applicability will become apparent from the description provided herein. It should be understood that the description and specific examples are intended for purposes of illustration only and are not intended to limit the scope of the present disclosure.

In one embodiment, an imaging system is provided including a reference marker, a medical device, an imaging array, a computer system, and a display system. The imaging system is for use in medical operations involving an operational area of the patient. The reference marker is fixed in a position within the operational area. The medical device is moveable within the operational area and includes a comparison marker. The imaging array observes the position of the reference marker and a position of the comparison marker within the operational area. The imaging array comprises at least two cameras. The computer system constructs a three-dimensional model of the operational area and defines the position of the reference marker within the operational area using at least two two-dimensional images of the operational area. The display system is in communication with the imaging array and the computer system. The display system creates a display of the three-dimensional model of the operational area and a position of the medical device within the operational area.

In another embodiment, a medical needle is provided including an elongate element, a housing, and a comparison marker. The medical needle is used alongside an imaging system in a medical operation involving an operational area of a patient. The imaging system includes a reference marker fixed in a position within the operational area, an imaging array observing the operational area, a computer system which constructs a three-dimensional model of the operational area and which defines the position of the reference marker within the operational area, and a display system in communication with the imaging array and the computer system. The display system creates a display of the three-dimensional model of the operational area. The elongate element extends from a proximal end to a distal end and has an increasing stiffness profile from the distal end to the proximal end. The housing is coupled to the proximal end of the elongate element. The comparison marker is coupled to the housing. As the comparison marker is observed by the imaging array while the elongate element is within the operational area, the display system is adapted to create the display further including a position of the elongate element within the operational area.

In yet another embodiment, a method of conducting a medical operation involving an operational area of a patient is provided, including fixing a reference marker in a position within the operational area, creating at least two two-dimensional X-ray images of the operational area, constructing a three-dimensional model of the operational area, defining the position of the reference marker within the operational area, moving a medical device within the operational area, observing the position of the reference marker and a position of a comparison marker within the observational area using an imaging array, and displaying the three-dimensional model of the operational area and the position of the medical device within the operational area on a display. The at least two two-dimensional X-ray images are used to construct the three-dimensional model of the operational area. The at least two two-dimensional X-ray images are also used to define the position of the reference marker. The comparison marker is coupled to the medical device. The imaging array includes at least two cameras. The display is created by a display system in communication with the imaging array and the computer system.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a perspective view of a first example of an imaging system including a fluoroscope, an imaging array, an operational area, reference markers, and a display system;

FIG. 2 illustrates a bottom plan view of a second example of the imaging system including an image intensifier, the imaging array, cameras, and the display system;

FIG. 3 illustrates a side plan view of a third example of the imaging system including the imaging array, cameras, the display system, and a display;

FIG. 4 illustrates a side plan view of a fourth example of the imaging system including the fluoroscope, the imaging array, the operational area, the reference markers, and the display system;

FIG. 5 illustrates a top plan view of a fifth example of the imaging system including the fluoroscope, the operational area, the imaging array, the display system, and a medical device;

FIG. 6 illustrates a perspective view of an example of the reference marker;

FIG. 7 illustrates a flow diagram of a sixth example of the imaging system including the fluoroscope, the operational area, a computer system, the imaging array, an accelerometer, the display system, the medical device, and the display;

FIG. 8 illustrates a side-by-side comparison of an example of a real operational area with an example of a dimensional operational area including reference markers and the medical device;

FIG. 9 illustrates a front plan view of a seventh example of the imaging system including the imaging array, the display system, and the display.

FIG. 10 illustrates a cross-sectional side view of an example of the medical device including an elongate element, a cannula, a reinforcing element, a housing, and a comparison marker attachment;

FIG. 11 illustrates a cross-sectional side view of a second example of the medical device including the elongate element, the cannula, the reinforcing element, and the housing;

FIG. 12 illustrates a side plan view of a third example of the medical device including the elongate element, the cannula, the reinforcing element, the housing, the comparison marker attachment, and comparison markers;

FIG. 13 illustrates a perspective view of a fourth example of the medical device including the elongate element, the cannula, the reinforcing element, the housing, the comparison marker attachment, and the comparison markers;

FIG. 14 illustrates a side plan view of a fifth example of the medical device including the housing, the comparison marker attachment, the comparison markers, and a sensor; and

FIG. 15 illustrates a flow diagram of operations to operate an imaging system during a medical procedure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

In one example, an imaging system is provided including a reference marker, a medical device, an imaging array, a computer system, and a display system. The imaging system is for use in medical operations involving an operational area of the patient. The reference marker is fixed in a position within the operational area. The medical device is moveable within the operational area and includes a comparison marker. The imaging array observes the position of the reference marker and a position of the comparison marker within the operational area. The imaging array comprises at least two cameras. The computer system constructs a three-dimensional model of the operational area and defines the position of the reference marker within the operational area using at least two two-dimensional images of the operational area. The display system is in communication with the imaging array and the computer system. The display system creates a display of the three-dimensional model of the operational area and a position of the medical device within the operational area.

One technical advantage of the systems and methods described below may be that the method described may use only a small number of two-dimensional X-ray images to create a three-dimensional model of the operational area, thereby reducing the amount of radiation to which a patient may be exposed. Furthermore, utilizing the three-dimensional model with an imaging system may reduce the need of fluoroscopy during the medical procedure, further reducing the X-ray exposure of the patient. Additionally, constructing the three-dimensional model from two-dimensional X-ray images may allow medical procedures to be conducted without on-site CT scanning equipment, allowing wider availability for medical procedures in more remote areas.

Yet another technical advantage of the systems and methods described below may be that the medical device described may have a small caliber and may also be reinforced, allowing it to be advanced through complex geometry of the operational area without being misdirected. Yet another technical advantage of the systems and methods described below may be that the medical device described may include comparison markers, allowing it to navigate complex geometry within the operational area and also to be observed within the body as it is being advanced to a treatment site.

Yet another technical advantage of the systems and methods described below may be that the equipment used for creating the two-dimensional X-ray images before the procedure and the equipment used to track the position of the medical device during the procedure may be integrated into a single imaging system. For example, a fluoroscope may be used to create the two-dimensional X-ray images and the imaging array which observes and tracks the medical device within the operational area may be coupled to the fluoroscope.

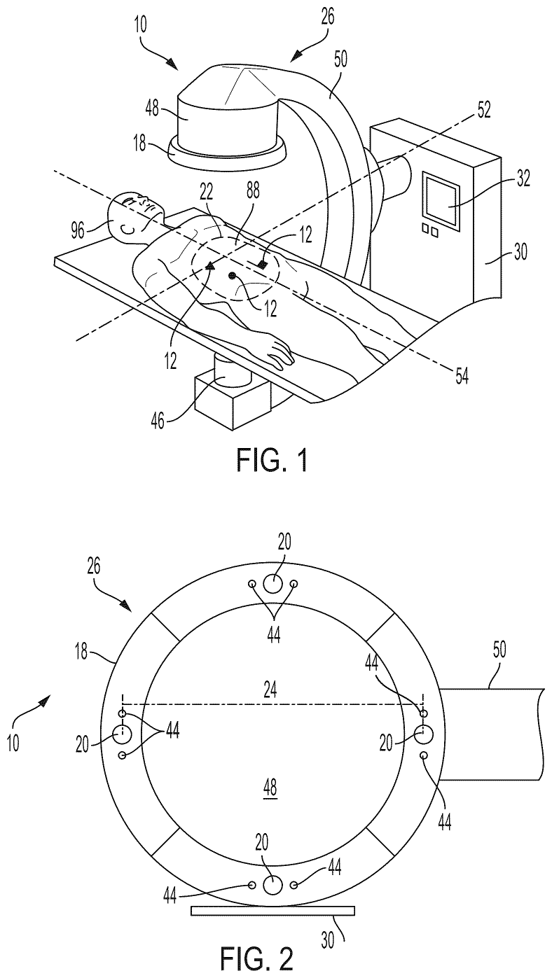

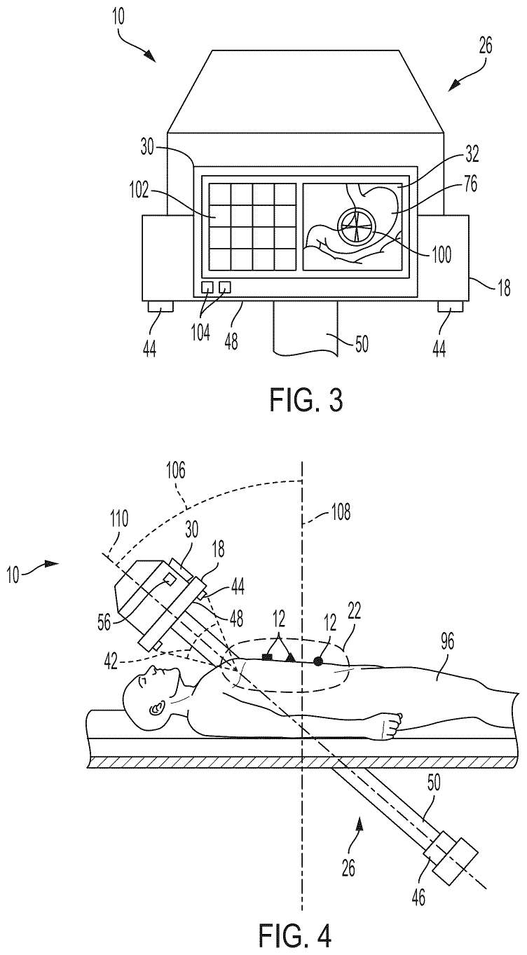

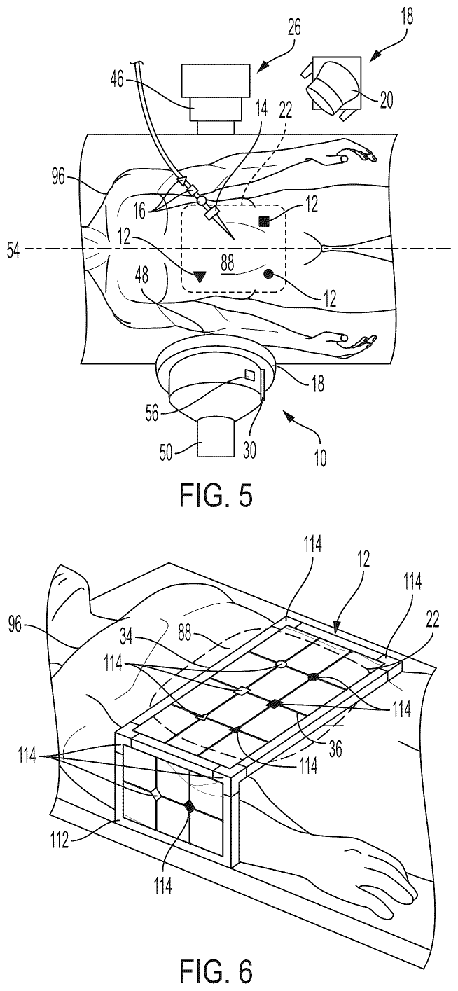

FIG. 1 illustrates an example of an imaging system 10 including a fluoroscope 26, reference markers 12 in an operational area 22, an imaging array 18, and a display system 30. The imaging system 10 may be any system used during a medical procedure which allows an operator to visualize a three-dimensional model (76 in FIG. 3) of the operational area 22 and track the movement of a medical device (14 in FIG. 5) within the operational area 22. The operational area 22 may be any portion of a body of a patient 96 to which treatment is to be performed. Examples of the operational area 22 may be a specific organ, a group of organs, or just a region of the body. The operational area 22 may include the area above and beneath an operating surface 88 if they contain tools, devices, and areas of the body used in the medical procedure. The operating surface 88 may be any portion of the body beneath which, the operator cannot see without visualization tools such as scopes, fluoroscopy, or other imaging techniques. Examples of the operating surface 88 may include the skin of the patient 96, a bone, or a tissue boundary.

The fluoroscope 26 may be any device which is capable of using radiation to create two-dimensional X-ray images of the operational area 22 before and during the medical procedure. Examples of the fluoroscope 26 may include a C-Arm fluoroscope, a stationary fluoroscope, or a portable, battery-operated fluoroscope. The fluoroscope 26 may include an X-ray generator 46, an image intensifier 48, and a C-arm 50. The X-ray generator 46 may be any component of the fluoroscope 26 capable of generating a beam of X-ray radiation. The image intensifier 48 may be any portion of the fluoroscope 26 capable of receiving X-ray radiation from the X-ray generator 46 and generating an X-ray image. The C-arm 50 may be any portion of the fluoroscope 26 which connects the X-ray generator 46 and the image intensifier 48. Examples of the C-arm 50 include a bracket or a curved structure. In some embodiments, the C-arm 50 extends around the patient 96 such that the X-ray generator 46 is beneath the patient 96 and the image intensifier 48 is above the patient 96.

The fluoroscope 26 may be moveable about a first axis 52 and a second axis 54. In some embodiments, the C-arm 50 may rotate about the first axis 52 to reposition the X-ray generator 46 and the image intensifier 48 to the top or bottom of the operational area of the patient 96. In such embodiments, the first axis 52 may extend through the C-arm 50. Alternatively, the C-arm 50 may translate along a circumference about the second axis 54 to reposition the X-ray generator 46 and the image intensifier 48 to the left or right side of the patient 96. In such embodiments, the second axis 54 may extend parallel to the length of the body of the patient 96.

For example, when generating two-dimensional X-ray images, the fluoroscope 26 may create a first image from directly below the operational area 22, then rotate about first axis 52 to create a second image on a first side of the operational area 22. The fluoroscope 26 may then translate along the circumference about the second axis 54 to take a third image on a second side of the operational area 22. The fluoroscope 26 may undergo further rotations and translations about the first axis 52 and the second axis 54 to take further X-ray images of the operational area 22.

For example, in one embodiment, the fluoroscope 26 may create an initial X-ray image while the X-ray generator 46 is in a horizontal position (0°). The fluoroscope 26 may then translate along the circumference about the second axis 54 and create between 4-20 X-ray images. For example, the fluoroscope 26 may create 7 images in a half-circumferential translation about the second axis 54, each image being spaced evenly apart (e.g. 0°, 30°, 60°, 90°, 120°, 150°, 180°). In another example, the fluoroscope 26 may create 18 images in a circumferential translation about the second axis 54, each image being spaced evenly apart (e.g. 0°, 20°, 40°, 60°, 80°, 100°, 120°, 140°, 160°, 180°, 200°, 220°, 240°, 260°, 280°, 300°, 320°, 340°).

Alternatively, or in addition to circumferential movement, images can be acquired with a C-arm of the fluoroscope 26 tilted (e.g., cranial/caudal tilt) relative to the primary movement path, for instance, tilted up to +/−45 degrees or more, to obtain additional views. Furthermore, images may be obtained over a limited arc circumferentially, such as within a 90-degree arc, but this limited arc acquisition may be supplemented with multiple other views obtained by tilting the C-arm at various points within that arc. The combination of views, whether purely circumferential, tilted, acquired over a limited arc, or a combination thereof, should be sufficient to allow the computer system 28 to construct the three-dimensional model 76. In some embodiments, these images may be converted into mesh-like images before being assembled in the three-dimensional model of the operational area.

The reference markers 12 may be any device which may be fixed in a position in the operational area 22 and which may be visible both in X-ray images and in visual observation of the operational area 22. Examples of the reference markers 12 may include fiducials, beacons, or positional anchors. The reference markers 12 may be radiopaque and may be made from a material such as gold, tantalum, stainless steel, or barium sulfate. Each reference marker 12 may have a shape which is visually distinguishable from any of the other reference markers 12. For example, where three reference markers 12 are present, they may have triangular, square, and circular shapes, or may have three-dimensional shapes such as a cube, cylinder, or pyramid. Each of the reference markers 12 may have a different color which is distinguishable by the imaging array 18. In some embodiments, the imaging system 10 may include three reference markers 12 adapted to be fixed in three respective positions within the operational area 22 such that each of the three reference markers 12 is spaced apart from each of the other reference markers 12.

The imaging array 18 may be any portion of the system which may observe the operational area 22. Examples of the imaging array 18 may include a camera, an infrared imager, a light detection and ranging system (LIDAR), or an array of several of the aforementioned devices. As illustrated in FIG. 1 the imaging array 18 may be coupled to the image intensifier 48. However, in some embodiments where the X-ray generator 46 is in view of the operational area 22, the imaging array 18 may be coupled to the X-ray generator 46. In other embodiments, the imaging array 18 may be separated from the fluoroscope 26 entirely.

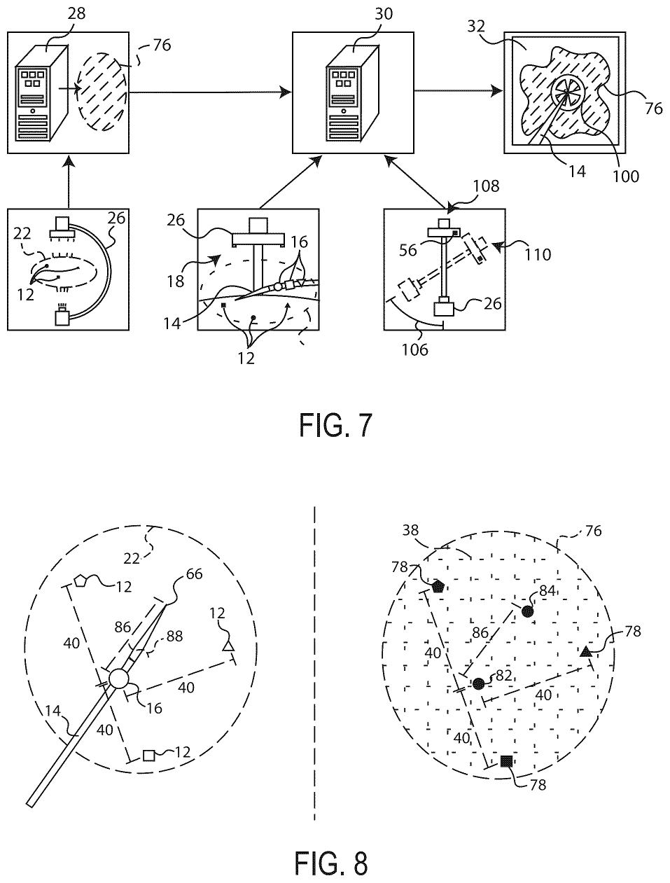

The display system 30 may be any portion of the imaging system 10 which is in communication with a computer system (28 in FIG. 7) and the imaging array 18 and which creates a display 32 visualizing the operational area 22 for the operator. The display system 30 may be coupled to the fluoroscope 26 or may be separated from the fluoroscope 26. The display system 30 be electrically coupled to the imaging array 18 and the computer system 28 or may be electrically separated. For example, in some embodiments the display system 30 may be in communication with the imaging array 18 and the computer system 28 wirelessly or over a computer network. Similarly, the display 32 may be physically and electrically coupled to the display system 30 or may be separate and wirelessly in communication with the display system 30. For example, as illustrated in FIG. 1, the display system 30 may be coupled to the fluoroscope 26 and the display 32. Alternatively, in other embodiments, the display 32 is on a separate computer tablet is created by the display system 30 and transmitted to the computer tablet.

FIG. 2 illustrates a bottom plan illustration of the image intensifier 48 including the imaging array 18 and the display system 30. The imaging array 18 may include at least two cameras 20 facing the operational area 22. The cameras 20 may be any device which receives light from the operational area 22 and sends data to the display system 30. Examples of the cameras 20 may include optical cameras, infrared cameras, or ultraviolet cameras. The cameras 20 may be separated from each other by at least 6 inches to create stereoscopic vision of the operational area 22. The cameras 20 of the imaging array 18 may be spaced apart from each other such that an angular separation (42 in FIG. 4) between the cameras 20 from the operating surface 88 within the operational area 22 is at least 5 degrees. Additional cameras 20 may be included in the imaging array 18 to further enhance the parallax between cameras 20 and more accurately perceive depth of objects within the operational area 22 as well as provide redundancy and resilience if one or more cameras' views of the reference markers 12 is occluded.

In some embodiments, the imaging array 18 may include one or more light sources 44 to enhance the operability of the cameras 20. The light source 44 may be any device which projects light onto the operational area 22. Examples of the light source 44 may include a white light, an ultraviolet light, a laser ranging system, or an infrared light. For example, in some embodiments, the light source 44 may illuminate the operational area 22 with infrared light. In such an embodiment, the reference markers 12 and the comparison markers (16 in FIG. 5) may be made of a material which is reflective of infrared light, and the cameras 20 may be adapted to observe infrared light specifically. In such an embodiment, the imaging array 18 may adequately observe the operational area 22 without over-saturating the operational area 22 with optical light. In some embodiments, the imaging array 18 may include multiple types of light sources 44 to be able to adapt the illumination of the operational area 22. In embodiments wherein the light source is a laser ranging system, the distance data collected by the light source may be sent to the computer system 28 to further enhance the creation of the three-dimensional model 76 of the operational area 22. Similar effects could be achieved by including stereophotogrammetric cameras in the imaging array 18.

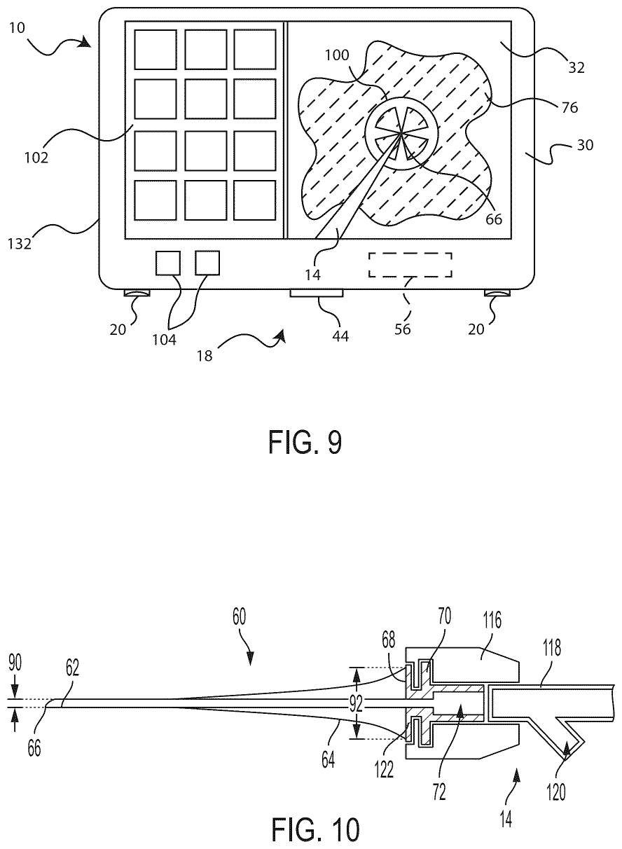

FIG. 3 illustrates a side plan view of the image intensifier 48. In some embodiments, the display 32 and display system 30 may be coupled to the image intensifier 48 to be easily visible to an operator during the medical procedure. The display 32 may illustrate the three-dimensional model 76 of the operational area 22 and a position of a medical device (14 in FIG. 5) within operational area 22. For example, in one embodiment, the display 32 may include cross-hairs 100 which indicate a distal end (66 in FIG. 9) of the medical device 14 in relation to the three-dimensional model 76 of the operational area 22. As the medical device 14 moves through the operational area 22, the display 32 may update the position of the medical device 14 relative to the three-dimensional model 76. Through such a display 32, an operator may accurately know the position of the medical device 14 within the geometry of the operational area 22, even if below the operating surface 88, and precisely advance the medical device 14 to a treatment area.

FIG. 4 illustrates another side plan view of the fluoroscope 26 and the operational area 22 of the patient 96. As discussed with respect to FIG. 1, in some embodiments, the fluoroscope 26 may be rotatable about the first axis 52 to move the fluoroscope 26 and the imaging array 18 from a first arrangement 108 to a second arrangement 110. In the first arrangement 108, the imaging array 18 may have a first view of the operational area 22 and the reference markers 12, while in the second view of the operational area 22 and the reference markers 12. The movement 106 of the imaging array 18 may be tracked by an accelerometer 56 and sent to the computer system 28 to calibrate the imaging system 10 and to enhance the depth of field of the stereoscopic view of the operational area 22 by the imaging array 18.

The accelerometer 56 may be coupled to the imaging array 18 or to the fluoroscope 26. The accelerometer 56 may be any device which tracks the movement 106 of the imaging array 18 to provide positional data of the imaging array 18 to the computer system 28 while the imaging array 18 is in the first arrangement 108 and the second arrangement 110. Examples of the accelerometer 56 may include a piezoelectric accelerometer, a capacitive accelerometer; or a microelectromechanical systems (MEMS) accelerometer. The accelerometer 56 may be mounted on the imaging array 18, the display system 30, or the fluoroscope 26, or may be integrated into those components.

FIG. 5 illustrates a top view of the fluoroscope 26 over the operational area 22 along with the medical device 14. As discussed with respect to FIG. 1, in some embodiments, the fluoroscope 26 may be rotatable about the second axis 54 to move the fluoroscope 26 and the imaging array 18 from a first arrangement 108 to a second arrangement 110. Similar to rotation about the first axis 52, the movement 106 of the imaging array 18 may be tracked by an accelerometer 56 and sent to the computer system 28 to calibrate the imaging system 10 and to enhance the depth of field of the stereoscopic view of the operational area 22 by the imaging array 18.

In some embodiments, one of the cameras 20 may be part of the imaging array 18 which is not coupled to the fluoroscope 26. For example, one of the cameras 20 may be in a fixed position as shown in FIG. 5 while the cameras 20 coupled to the fluoroscope 26 move from rotation. Such a configuration may be advantageous in calibrating the imaging system 10 and in enhancing the depth of field of the stereoscopic view of the operational area 22 by the imaging array 18.

The medical device 14 may be any equipment moved within the operational area 22 during the medical procedure. Examples of the medical device 14 may include a needle, a catheter, a trocar, an introducer, a scalpel, or an ocular scope. The medical device 14 may include one of more comparison markers 16 observable by the imaging array 18. Examples of the comparison markers 16 may include indicators, beacons, or guides. In some embodiments, the comparison markers 16 may be radiopaque and may be made from a material such as gold, tantalum, stainless steel, or barium sulfate. However, as the medical device 14 may not be present during creation of the two-dimensional X-ray images, in other embodiments, the comparison markers 16 may not be radiopaque and may be made from a material such as polyethylene, polypropylene, or glass. Each comparison marker 16 may have a shape which is visually distinguishable from any of the other comparison markers 16. For example, where three comparison markers 16 are present, they may have triangular, square, and circular shapes, or may have three-dimensional shapes such as a cube, cylinder, or pyramid. Each of the comparison markers 16 may have a different color which is distinguishable by the imaging array 18. Each of the comparison markers 16 may be spaced apart from each of the other comparison markers 16 such that the position and orientation of the medical device 14 may be observed.

FIG. 6 includes a perspective view of the operational area 22 including the reference marker 12 in the form of a cage extending across a portion of the operational area 22. In some embodiments, the reference marker 12 cage may extend across the operational area 22 and provide many reference points for the computer system 28 to use in constructing the three-dimensional model 76 of the operational area 22. For example, the cage may include a boundary 34 encompassing a large portion of the operational area. Within the boundary 34, a grid 36 may extend across the cage to provide reference lines to construct the three-dimensional model 76 of the operational area 22 and to enhance the observation of the imaging array 18. In some embodiments, the cage may be made of a non-radiopaque material such as polyethylene or polypropylene while specific radiopaque reflective points 114 are positioned at key positions around the cage, such as the corners of the boundary 34 or at intersections within the grid 36. The reflective points 114 may also be visually distinguishable by the imaging array 18 by having unique colors or shapes. In other embodiments, the entire cage may be radiopaque.

In one example, the reference marker 12 cage may take the form of a simple scalene triangle with radiopaque identifiers positioned at each vertex. In such an example, the boundary 34 in such an embodiment may be a semi-rigid tape with adhesive to affix the cage to the patient 96. Such a boundary would fix the reference marker 12 in place relative to the patient 96 but would also maximize the ability of instruments to move within the operational area 88.

The cage may also include a depth portion 112 angularly offset from the rest of the cage. For example, a portion of the cage may extend across an abdomen or a back of a patient 96 while the depth portion 112 of the cage may extend downward along the side of the patient 96. The depth portion 112 may also have a boundary 34 and a grid 36 and may include reflective points 114 which are radiopaque and also visually distinguishable by the imaging array 18. The depth portion 112 may enhance the ability of the computer system 28 to construct a three-dimensional model 76 of the operational area 22 and may enhance the ability of the imaging array 18 to perceive the position of the medical device 14 within the operational area 22.

FIG. 7. Illustrates a flow diagram of the operation of the imaging system 10. In some embodiments, two-dimensional X-ray images of operational area 22 and the reference markers 12 are taken by the fluoroscope 26. The two-dimensional X-ray images are sent to the computer system 28 for construction of a three-dimensional model 76 of the operational area 22 including the position of the reference markers 12 within the operational area 22. In some embodiments, the accelerometer 56 may also send data to the computer system 28 such that the arrangement for each two-dimensional X-ray image with respect to the operational area 22 may be used in the construction of the three-dimensional model 76.

The computer system 28 may be any component which can process two-dimensional images to construct a three-dimensional model. Examples of the computer system 28 may include a processing unit physically incorporated into the imaging array 18 or display system 30 or an off-site server connected to the imaging array 18 and display system 30 through a local network or the internet. An internet-based computer system 28 may be advantageous to remote facilities who may not be able to afford the purchase and upkeep of complicated computing equipment.

In some embodiments, the computer system 28 may incorporate artificial intelligence computing methods to construct the three-dimensional model 76. For example, three-dimensional morphable models (3DMM), deep learning-based depth estimation, generative adversarial networks, self-supervised neural networks, and synthetic CT methods may all be used to quickly construct the three-dimensional model 76 from the two-dimensional X-ray images created by the fluoroscope 26. To limit the radiation exposure to the patient, the computer system 28 may construct the three-dimensional model 76 from no more than four two-dimensional X-ray images. However, in some circumstances and using some artificial intelligence processing methods, an adequate three-dimensional model 76 may be constructed from only a single two-dimensional X-ray image. In other embodiments, as many as 20 two-dimensional X-ray images may be used for cone-beam CT reconstruction techniques, depending on the accuracy needed for the procedure and the complexity of the operational area. 22. Using the methods described above, fewer two-dimensional X-ray images may be needed to create an adequate three-dimensional model 76 as compared to traditional CT. Once constructed, the computer system 28 may send the three-dimensional model 76 of the operational area 22 to the display system 30.

During the medical procedure, the imaging array 18 may observe the operational area 22. The medical device 14 may also be introduced into the operational area 22. The position of the reference markers 12 and comparison markers 16 may be observed from each camera 20 in stereoscopic view and sent to the display system 30. Furthermore, the accelerometer 56 may also send data to the display system 30 such that the arrangement for the image array's 18 observation of the operational area 22 may be used in the construction of the display 32.

Once the display system 30 has received the constructed three-dimensional model 76 of the operational area 22, the accelerometer 56 data for the imaging array 18 and the fluoroscope 26, as well as the positional information of the reference markers 12 and the comparison markers 16, the display system 30 may align the positions of the reference markers 12 in the three-dimensional model 76 with the positions of the reference markers 12 observed by the imaging array 18 to create a full model including the position of the medical device 14 within the operational area 22. The display system 32 may then create a display 32 including the three-dimensional model 76 of the operational area 22 as well as the position of the medical device 14 within the operational area 22.

FIG. 8 illustrates a side-by-side comparison view of an example of the operational area 22 alongside a positional view of the same operational area 22. In some embodiments, the computer system 28 may reduce the reference markers 12 to reference marker positions 78 within a three-dimensional grid 38 of the operational area 22. For clarity, FIG. 8 only shows a two-dimensional simplification of this three-dimensional grid 38. As the comparison markers 16 are observed within the operational area 22, the display system 30 may determine the positions 82 of the comparison markers 16 by comparing the relative distance 40 of the reference marker positions 78 from the comparison markers 16.

When multiple comparison markers 16 are present and the arrangement of the comparison markers 16 is known by the display system 30, the orientation 126 of the medical device 14 may also be determined. Additionally, when the distance 86 from the comparison markers 16 to the distal end 66 of the medical device 14 is known, a position 84 of the distal end 66 may also be determined. With such information known, the display system 30 may, for example, create a display 32 from the perspective of the medical device 14 within the three-dimensional model 76 of the operational area 22 looking distally toward the distal end 66. As shown in FIG. 3, the position 84 of the distal end 66 may be marked with a cross-hair 100 symbol.

FIG. 9 illustrates another embodiment of the imaging system 10 including the imaging array 18, the display system 30, and the display 32 in an integrated unit. In such an embodiment, the cameras 20 may be mounted on the exterior of a tablet-like body 132. The display system 30 and accelerometer 56 may be contained within the body 132. The body 132 may be mounted on the fluoroscope 26 before the two-dimensional X-ray images are created. The accelerometer 56 may then record the position of the fluoroscope 26 while creating the X-ray images based on acceleration data as the fluoroscope 26 moves. This data may be sent to a website or off-site server for the computer system 28 to construct the three-dimensional model 76 of the operational area 22. The completed three-dimensional model 76 may then be sent back to the display system 30. During the medical procedure the imaging system 10 may remain mounted to the fluoroscope 26 to observe the operational area 22 and create the display 32. In some embodiments, the display system 30 may be in communication with an external monitor and may transmit the display 32 to that other monitor.

The display system may include one or more calibration buttons 104 which may be pressed once the imaging system 10 has been mounted on the fluoroscope 26. The calibration button 104 may ready the imaging system 10 for operation by zeroing out the accelerometer 56 data once the imaging system 10 has been mounted. Furthermore, just before the medical procedure, the calibration button 104 may be pressed to calibrate the cameras 20 and adjust their lens for optimal light reception.

FIG. 10 illustrates a cross-sectional side view of an example of the medical device 14. In some embodiments, the medical device 14 may be a needle including an elongate element 60, a housing 70, a reinforcing clip 116, and a proximal attachment 118. The elongate element 60 may be any portion of the medical device 14 which is adapted to be advanced through the operational area 22 to the treatment site. Examples of the elongate element 60 may include a needle, a catheter, or an introducer. The elongate element 60 may extend from a proximal end 68 to the distal end 66 of the medical device 14. The elongate element 60 may include a cannula 62 and a reinforcing member 64. The cannula 62 may be portion of the elongate element 60 which is hollow and adapted to deliver treatment fluid to the treatment site. The cannula 62 may be narrow to allow for flexibility near the distal end 66 of the medical device 14. The perimeter of the cannula 62 may define a passage extending through the distal end 66, the proximal end 68, and through at least a portion of the housing 70.

The reinforcing member 64 may be any portion of the elongate element 60 which surrounds at least a portion of the cannula 62. The reinforcing member 64 may have an increasing stiffness profile extending distally from the proximal end 68. In some embodiments, the increasing stiffness profile of the reinforcing member 64 may result from the reinforcing member 64 having a distal thickness 90 which is comparable to a thickness of the cannula 62 and which increases toward a proximal thickness 92 at the proximal end 68. The combination of the cannula 62 and the reinforcing member 64 allow the elongate element 60 to also have an increasing stiffness profile from the distal end 66 of the medical device 14 to the proximal end 68 of the elongate element 60. The reinforcing member 64 may be coupled to the housing 70 at the proximal end 68 of the elongate element 60. The reinforcing member 64 may be made of any sufficiently rigid material such as epoxy resin or a ceramic material.

The housing 70 may be any portion of the medical device 14 which extends proximally from the elongate element 60. The passage 72 may extend through a portion of the housing 70 to allow connection to a treatment fluid source. The housing 70 may include a distal wall 122 adapted to be coupled to the reinforcing member 64 of the elongate element 60 to further stiffen the elongate element 60. The distal wall 122 of the housing 70 may have a diameter which is at least as large as the proximal thickness 92 of the reinforcing member 64. The housing 70 and the elongate element 60 may be further reinforced by a reinforcing clip 116 which may surround the housing 70. As an example, the reinforcing clip 116 may be two half shells which, when connected together, encompass the housing 70. In some embodiments, the reinforcing clip 116 may also encompass a portion of the reinforcing member 64.

The proximal attachment 118 may be any portion of the medical device 14 which is coupled to the housing 70 and extends proximally from the housing 70. The proximal attachment 118 may include a luer fitting 120 to couple a treatment fluid source to the passage 72.

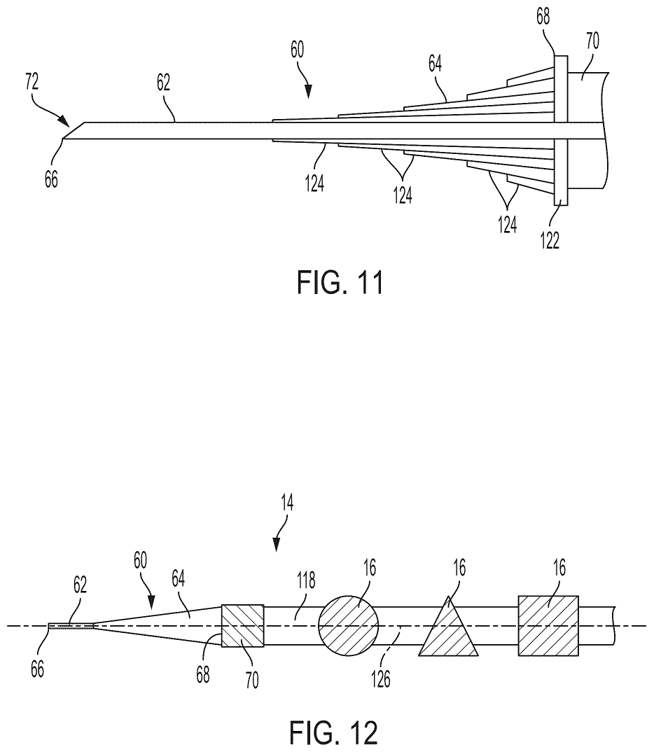

FIG. 11 illustrates a cross-sectional side view of another embodiment of the medical device 14. In some embodiments the reinforcing member 64 may be made from a number of tubes 124 nested over one another to create the increasing stiffness profile of the reinforcing member 64. The overlap between the tubes 124 may be staggered as shown in FIG. 11 to create an increasing thickness extending proximally. The tubes 124 may be micro-welded together or may be bonded using epoxy or a ceramic overlay. The increasing stiffness profile of the reinforcing member 64 may depend on the medical procedure for which it is being used. For example, in procedures where the medical device may be advanced deep below the operating surface 88 to reach the treatment site, such as in endoscopic procedure, the reinforcing member 64 may have an increasing stiffness profile which gradually increases proximally. Alternatively, in procedures where the medical device may be advanced deep below the operating surface 88 to reach the treatment site, such as spinal injections, the reinforcing member 64 may have an increasing stiffness profile which quickly increases proximally.

FIG. 12 illustrates another example of the medical device 14 including the comparison markers 16 coupled to the proximal attachment 118. The comparison markers 16 may be attached to either the housing 70 or the proximal attachment 118. However, the housing 70 and proximal attachment 118 extending distally from the comparison markers 16 must be rigid to ensure that the display system 30 accurately calculates the position of the medical device 14 within the operational area 22. In the embodiment shown in FIG. 12, the comparison markers 16 are arranged in a linear arrangement. The linear arrangement of the comparison markers 16 may define the orientation 126 of the medical device 14 for the display system 30.

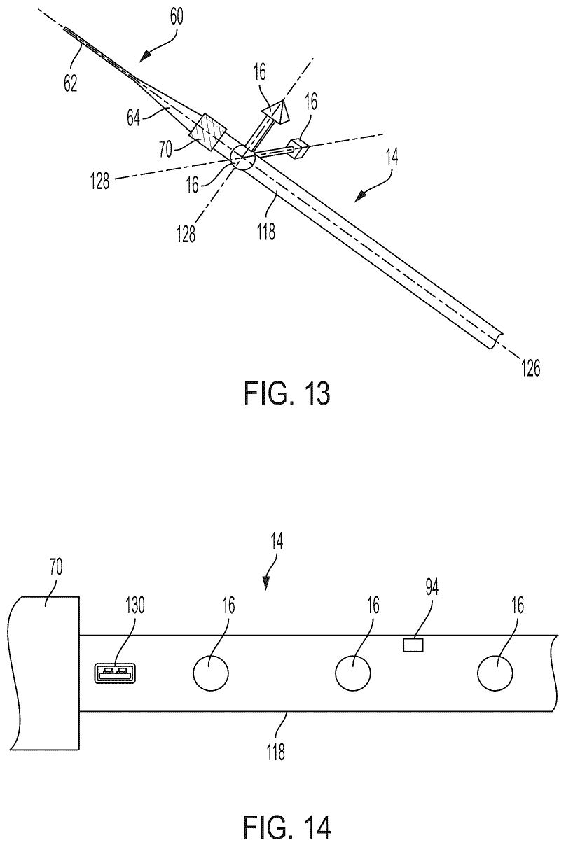

FIG. 13 illustrates another example of the medical device 14 including the comparison markers 16 coupled to the proximal attachment 118. In other embodiments, the comparison markers 16 may be spaced apart from each other along different axis 128 from the orientation 126 of the medical device 14. In such an embodiment, the orientation 126 of the medical device 14 may still be accurately calculated as long as the specifications and measurements of the medical device are transmitted to the display system 30.

FIG. 14 illustrates another example of the medical device 14 include the comparison markers 16 arranged along the proximal attachment 118. In such an embodiment, the comparison markers 16 may be light-emitting diodes (LED) adapted to broadcast a code to the imaging array 18. The code may be observed by the cameras 20 of the imaging array 18 and processed by the display system 30. For example, the comparison marker 16 may transmit a code transmitting the physical characteristics of the medical device 14 to the display system 30, such as the relative position of the distal end 66 of the medical device 14 relative to the position of the comparison markers 16, or the relative position of the comparison markers 16 to each of the other comparison markers 16.

In some embodiments, the medical device 14 may include a positional sensor 94 in communication with the light-emitting diode comparison markers 16. The positional sensor 94 may be any device which is capable of recording positional or orientation data of the medical device 14. Examples of the positional sensor 94 may include a piezo-electric torsion sensor, an accelerometer, or a gyroscope. In some embodiments, the positional sensor 94 may send data regarding the position, orientation, or bending of the medical device 14 to the comparison marker 16, which may then be transmitted as a code to the imaging array 18 and processed by the display system 30. In such embodiments, the medical device 14 may also include an electrical/data port 130, such as a Universal Serial Bus (USB) port, coupled to the positional sensor 94 and comparison markers 16 to provide electrical power to those components.

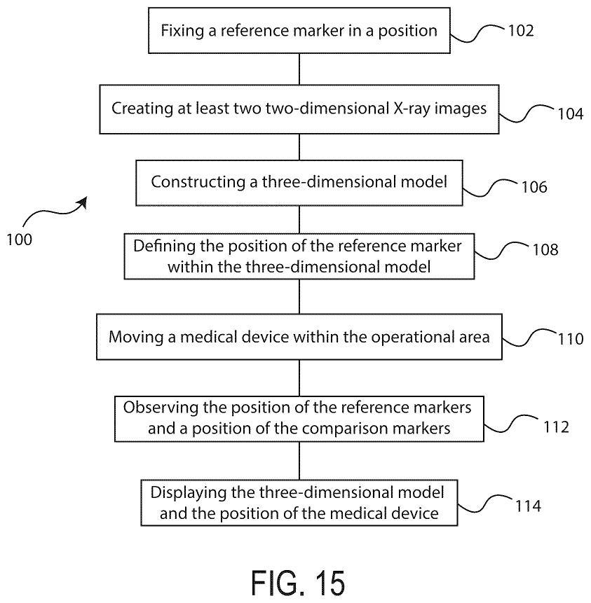

FIG. 15 illustrates a flow diagram of operations to conducting a medical operation involving the operational area 22 of the patient 96. The operations may include fewer, additional, or different operations than illustrated in FIG. 8. Alternatively or in addition, the operations may be performed in a different order than illustrated.

The method (100) includes fixing the reference marker 12 in the position within the operational area 22 (102). Additionally, at least two two-dimensional X-ray images of the operational area 22 are created (104). The three-dimensional dimensional model 76 of the operational area 22 is constructed from the at least two two-dimensional X-ray images using the computer system 28 (106). The position of the reference markers 12 within the operational area 22 are defined from the at least two two-dimensional X-ray images using the computer system 28 (108). The medical device 14 is moved within the operational area including the comparison markers 16 (110). The positions of the reference markers 12 and the positions of the comparison markers 16 within the operational area 22 are observed by the imaging array 18 using at least two cameras 20 (112). The three-dimensional model 76 of the operational area 22 and the position of the medical device 14 within the operational area 22 are displayed on the display 32 using the display system 30 in communication with the imaging array 18 and the computer system 28 (114).

In some embodiments, moving the medical device 14 within the operational area 22 may include advancing the medical device 14 in the form of a trocar and introducer having known geometry. Once the medical device 14 has been advanced close enough to the treatment site, the introducer may be removed, leaving the hollow trocar which may be advanced to the treatment site under fluoroscopy.

In addition to the advantages that have been described, it is also possible that there are still other advantages that are not currently recognized but which may become apparent at a later time. While various embodiments have been described, it will be apparent to those of ordinary skill in the art that many more embodiments and implementations are possible. Accordingly, the embodiments described herein are examples, not the only possible embodiments and implementations.

We claim:

1. An imaging system for use in medical operation involving an operational area of a patient, comprising:
a reference marker adapted to be fixed in a position within the operational area;
a medical device adapted to be moved within the operational area, wherein the medical device comprises a comparison marker;
an imaging array adapted to observe the position of the reference marker and a position of the comparison marker within the operational area, wherein the imaging array comprises at least two cameras;
a computer system adapted to, from two-dimensional X-ray images of the operational area, construct a three-dimensional model of the operational area and define the position of the reference marker within the operational area; and
a display system in communication with the imaging array and the computer system, wherein the display system is adapted to, using the position of the reference marker and the position of the comparison marker, create a display of the three-dimensional model of the operational area and a position of the medical device within the operational area.

2. The imaging system of claim 1, comprising three reference markers adapted to be fixed in three respective positions within the operational area.

3. The imaging system of claim 2, wherein each of the three reference markers has a shape which is visually distinguishable from any of the other three reference markers.

4. The imaging system of claim 1, wherein the reference marker comprises a cage extending across a portion of the operational area.

5. The imaging system of claim 1, wherein the medical device comprises three comparison markers spaced apart from each of the other three comparison markers such that, from observation of the three comparison markers by the imaging array, the display system is adapted to create the display further comprising an orientation of the medical device.

6. The imaging system of claim 1, wherein each of the at least two cameras is spaced apart from the other of the at least two cameras such that an angular separation between the at least two cameras from an operating surface within the operational area is at least 5 degrees.

7. The imaging system of claim 1, further comprising an infrared light source adapted to illuminate the operational area, wherein:
the reference marker is reflective of infrared light;
the comparison marker is reflective of infrared light; and
the at least two cameras are adapted to observe infrared light.

8. The imaging system of claim 1, wherein at least one of the at least two cameras is moveable from a first arrangement having a first view of the operational area to a second arrangement having a second view of the operational area.

9. The imaging system of claim 8, further comprising a moveable fluoroscope comprising an X-ray generator and an image intensifier, wherein:
at least one of the at least two cameras is coupled to the image intensifier; and
the moveable fluoroscope is adapted to create the two-dimensional X-ray images.

10. The imaging system of claim 9, further comprising an accelerometer coupled to the moveable fluoroscope, wherein the accelerometer is in communication with the display system such that the display system is adapted to, from acceleration data from the accelerometer, determine an arrangement of at least one of the at least two cameras coupled to the image intensifier.

11. The imaging system of claim 9, wherein the display is arranged on the moveable fluoroscope.

12. A medical device for use alongside an imaging system in a medical operation involving an operational area of a patient, the imaging system comprising a reference marker fixed in a position within an operational area, an imaging array adapted to observe the operational area, a computer system adapted to construct a three-dimensional model of the operational area and define the position of the reference marker within the operational area, and a display system in communication with the imaging array and the computer system adapted to create a display of the three-dimensional model of the operational area, the medical needle comprising:
an elongate element extending from a proximal end to a distal end of the medical device, wherein the elongate element has an increasing stiffness profile from the distal end to the proximal end;
a housing coupled to the proximal end of the elongate element; and
a comparison marker coupled to the housing, wherein the comparison marker is adapted to be observed by the imaging array while the elongate element is within the operational area such that, while the elongate element is within the operational area, the display system is adapted to create the display further comprising a position of the elongate element within the operational area.

13. The medical needle of claim 12, wherein the elongate element comprises:
a cannula extending from the distal end of the medical device to the proximal end and defining a passage extending through the distal end, the proximal end, and the housing; and
a reinforcing member surrounding at least a portion of the cannula and coupled to the housing at the proximal end, wherein the reinforcing member has a thickness which increases toward the proximal end.

14. The medical needle of claim 12, further comprising three comparison markers adapted to be observed by the imaging array while the elongate element is within the operational area, wherein each of the three comparison markers are spaced apart from each of the others of the three comparison markers such that, while the elongate element is within the operational area, the display system is adapted to create the display comprising the position and an orientation of the elongate element within the operational area.

15. The medical needle of claim 12, wherein:
the comparison marker comprises a light-emitting diode (LED) adapted to broadcast a code indicating a relative position of the distal end of the medical device relative to the position of the comparison marker;
the imaging array is adapted to observe the code; and
the display system is adapted to process the code.

16. The medical needle of claim 12, wherein:
the comparison marker comprises a positional sensor and a light-emitting diode (LED);
the positional sensor is adapted to determine the orientation of the distal end of the elongate element;
the light-emitting diode is adapted to broadcast a code indicating the orientation of the distal end of the elongate element;
the imaging array is adapted to observe the code; and
the display system is adapted to process the code.

17. A method of conducting a medical operation involving an operational area of a patient, comprising:
fixing a reference marker in a position within an operational area;
creating at least two two-dimensional X-ray images of the operational area;
constructing a three-dimensional model of the operational area from the at least two two-dimensional X-ray images using a computer system;
defining the position of the reference marker within the operational area from the at least two two-dimensional X-ray images using the computer system;
moving a medical device within the operational area, wherein the medical device comprises a comparison marker;
observing the position of the reference marker and a position of the comparison marker within operational area using an imaging array comprising at least two cameras; and
displaying the three-dimensional model of the operational area and the position of the medical device within the operational area on a display using a display system in communication with the imaging array and the computer system.

18. The method of claim 17 wherein constructing a three-dimensional model of the operational area comprises the computer system using no more than four two-dimensional X-ray images of the operational area.

19. The method of claim 17, wherein:
the imaging array is coupled to an image intensifier of a fluoroscope; and
the fluoroscope is used in creating the at least two two-dimensional X-ray images.

20. The method of claim 19, further comprising:
observing the reference marker while the imaging array is in a first arrangement having a first view of the operational area;
moving the fluoroscope such that the imaging array is in a second arrangement; and
observing the reference marker while the imaging array is in the second arrangement having a second view of the operational area.
